(19) United States
(12) Patent Application Publication
(10) Pub. No.: US 2023/0190651 A1
(43) Pub. Date: Jun. 22, 2023

(54) Topical Aerosol Foams

(71) Applicant: ARCUTIS BIOTHERAPEUTICS, INC., Westlake Village, CA (US)

(72) Inventor: David W. OSBORNE, Fort Collins, CO (US)

(73) Assignee: ARCUTIS BIOTHERAPEUTICS, INC., Westlake Village, CA (US)

(21) Appl. No.: 18/146,112

(22) Filed: Dec. 23, 2022

Related U.S. Application Data
(60) Provisional application No. 63/371,967, filed on Aug. 19, 2022.
(60) Provisional application No. 63/291,178, filed on Dec. 17, 2021.

(51) Int. Cl. A61K 9/12; A61K 9/107; A61K 31/496; A61K 31/4164; A61K 45/06; A61K 31/365; A61K 31/57; A61K 47/10

(52) U.S. Cl. CPC A61K 9/12; A61K 9/107; A61K 31/496; A61K 31/4164; A61K 45/06; A61K 31/365; A61K 31/57; A61K 47/10

(57) ABSTRACT

The present invention is directed to an aerosol foam composition comprising an active pharmaceutical ingredient (API) with low water solubility an emulsifier blend containing cetearyl alcohol, dicetyl phosphate, and ceteareth-10 phosphate and a hydrocarbon propellant. The aerosol foam composition is preferably an oil in water emulsion. The propellant is a mixture of liquefied hydrocarbon gases preferably a propane/isobutane/butane blend. The hydrocarbon propellant results in an aerosol foam which is stable, has consistent physical properties, excellent aesthetics, and no discernable API degradation after long term or accelerated storage conditions.

### FIELD OF THE INVENTION

The present invention is directed to an oil in water emulsion aerosol foam composition having an alkyl phosphate anionic surfactant or blend of alkyl phosphate surfactants as the emulsifier. More particularly, the invention pertains to a pharmaceutically acceptable emulsion aerosol foam composition comprising a pharmaceutically active agent which has low solubility in water, and an emulsifier blend of cetearyl alcohol, dicetyl phosphate and ceteareth-10 phosphate (also known as ceteth-10 phosphate). The aerosol foam is dispensed using a propellant blend.

### BACKGROUND OF THE INVENTION

Foam formulations have been used as a delivery system for cosmetic and pharmaceutical applications for several decades. Foams are preferred in some applications as they spread more easily and minimize rubbing. This is particularly advantageous when treating irritated skin or areas of skin which are covered by hair. Foam vehicles are preferred over ointments, gels and creams due to their ease of application, reduced stickiness and reduction in greasy feel. Patient preference for foam vehicles can lead to increased patient compliance and thus better treatment results.

There are different kinds of foam formulations which can be used to deliver active ingredients, including aqueous, hydroalcoholic, emollient, solvent based, petrolatum based and oil based foams. The different formulations have different characteristics, for example, emollient foams have a soothing, moisturizing effect and hydroalcoholic foams promote skin penetration and solubility of the active agents. The foam can be made using a propellant-free generation method such as the AIRSPRAY® foam dispenser (foam dispenser having a pump assembly which includes a liquid pump, an air pump and a common actuation part to simultaneously actuate the liquid pump and the air pump) or by using a pressurized container and a propellant.

Topical foams differ from ointments and creams in that the characteristics of the foam vehicle change. Prior to application, the foam formulation is usually in the form of a suspension or emulsion. When an aerosol foam formulation is discharged from the container, the liquid propellant volatilizes producing a semi-solid foam product that is expanded with gas phase propellant. If a propellant-free generation method is used, air is simultaneously pumped into the suspension or emulsion as the foam is being dispensed. The method used to generate the foam affects the foam appearance and stability.

Foams can be designed to have specific properties depending on factors such as the condition being treated, the area of the body being treated, and the active pharmaceutical ingredient in the formulation. The foam vehicle should have suitable stability so that it does not collapse after discharge from the container; low shear sensitivity so that only minimal rubbing is required; should be non-irritating, non-allergenic, and non-toxic; and should keep the active pharmaceutical agent solubilized.

Additionally, aerosol foam vehicles should contain a propellant that has minimal or no impact on the ozone layer of the atmosphere. Foams applied to the face or upper front torso should have minimal odor, since addition of fragrances to cover malodor is not preferred for pharmaceutical products. Foam structure is affected by various parameters including the type and concentration of the foaming agent, the viscosity of the liquid phase, the salt concentration, the temperature and the pH of the formulation.

Commercializable three phase pharmaceutical aerosols rely on surfactants that have limited solubility in both the internal oil and external aqueous phases. Upon shaking, the liquid hydrocarbon propellant mixes with the dispersed globules of the oil phase. The surfactants concentrate at the interface between the propellant/oil phase and the aqueous phase to form a thin film referred to as the “lamella.” It is the specific composition of this lamella that dictates the structural strength and general characteristics of the foam that forms when the liquid propellant in the internal phase transitions into a gas as soon as pharmaceutical emulsion leaves the pressurized environment of the aerosol canister. This liquid to gas phase transition forms the bubbles of the foam. Thick and tightly layered lamellae produce very structured foams that can support their weight. Stable foams cannot always be formed. The formation of a stable foam with the desired structure depends on many factors including but not limited to the specific components, the concentrations of the components, the viscosity of the liquid phase, and the propellant. These factors can be adjusted to produce stable foams with different structures such as expanding foams, quick breaking foams, stiff foams, and stout foams.

Foam collapse occurs when the pressure generated by the expanding internal gas phase exceeds the cohesive strength of the foam lamella. Three sources of an expanding internal gas phase are: 1) additional degassing of the lower vapor pressure liquid hydrocarbon propellant, 2) mechanical pressure (pushing) on the foam during rub-in, and 3) general warming of the foam to ambient (20-25° C.) or skin (32° C.) temperature following adiabatic cooling of the foam concentrate (70 psig) as it passes through the value and becomes a foam at ambient pressures. For a three phase emulsion pharmaceutical foam stabilized by alkyl phosphate surfactants, once the lamella is reduced to a single surfactant bilayer film, additional expansion of the internal gas phase will cause rupture of the foam cell and drainage of the product onto the skin surface.

Expanding foams and quick breaking foams are characterized by the expanding internal gas phase quickly causing the lamella to rupture to create visibly larger foam cells. The expanding foam will appear to initially “puff up” as the internal foam cells combine, but as the foam cells on the surface collapse, drainage of the product will deliver the active to the skin application site.

For stiff foams and stout foams, the fully degassed internal phase warmed to skin temperature does not generate sufficient pressure to exceed the cohesive strength of the lamella. The gas cells do not rupture until the added pressure of rub-in occurs. These more stable foams are ideal for scalp application because the foam can be placed against the scalp lesions in a “part of the hair” and then rubbed-in to break the foam and apply the active agent to diseased skin with minimum loss of product to the hair.

For three phase pharmaceutical emulsion foams to be commercially acceptable, the liquid hydrocarbon propellant must mix properly with the internal oil phase of the emulsion in order to form a foam when the product leaves the canister. If the propellant does not properly mix, then only a few foam cells will form when the liquid propellant transitions into gas and the majority of the propellant will transition into a gas outside of the emulsion when actuated. When shaken and immediately actuated through the valve an unacceptable “sputtering” foam that is non-uniform and very dense will be dispensed. Since propellant separate from the emulsion concentrate is dispensed even though the canister is properly shaken and inverted, the propellant will empty from the canister prior to expelling the entire amount of product. This foam product is commercially unacceptable due to incomplete emptying of the canister. For example if a prescription product is labeled to deliver 60 grams of foam (a one month supply), but the propellant is completely exhausted after delivering 48 grams of foam, the patient will not receive the full, prescribed treatment. Such a foam canister would fail the requirement for minimum delivered mass and would be recalled from the market.

Stable foams cannot always be formed. The formation of a stable foam with the desired structure depends on many factors including but not limited to the specific components, the concentrations of the components, the viscosity of the liquid phase, and the propellant. Any excipient added to the formulation that increases the solubility of the surfactant into external aqueous phase will destabilize the emulsion, reduce the stiffness of the lamella and result in the foam bubbles rupturing as soon as the liquid propellant transitions into a gas. In other words, a fluid emulsion would be dispensed from the canister that quickly flows away from the skin application site rather than forming a topical foam that remains at the application site until rub-in breaks the lamella and releases the drug product to the desired treatment site.

The cosmetic and pharmaceutical solvent diethylene glycol monoethyl ether (DEGEE) has been shown to reside in the aqueous continuous phase of emulsions and increase the solubility of surfactants and waxy components of the lamella into the continuous aqueous phase during the emulsification process (Hemandez, et al., Journal of Dispersion Science and Technology, Investigating the effect of transcutol on the physical properties of an O/W cream, Vol 41, No. 4, pp 600-606, 2020). The dramatic destabilization of a polyoxyethylene-20-stearyl ether and polyoxyethylene-2-stearyl ether emulsion when the DEGEE concentration was increased above 25% suggests that maintaining sufficiently thick and tightly layered lamellae to produce a stable foam in the presence of 25% or more DEGEE would be surprising.

Foam stability can be evaluated by determining the foam half-life. Foam half-life is the time required for half the volume of the liquid continuous phase of the foam product to drain. The shorter the half-life, the lower the foam stability. The desired foam half-life would be based in the intended use of the foam. For certain foam applications where the foam is applied over large areas of the body surface (for example self-tanning foams and sun screen foams), foam half-lives are preferred to be less than 30 seconds to minimize the application time. For the topical pharmaceutical foams of the present invention, a foam half-life of greater than 30 seconds is desirable and in some embodiments a foam half-life of greater than one minute is preferred.

Aerosol foams have been found to produce a stable foam which is suitable for topical application of active pharmaceutical ingredients (API). An aerosol foam formulation consists of two components: the product concentrate and the propellant. The product concentrate is the active drug combined with additional ingredients or co-solvents required to make a stable and efficacious product. The concentrate of a pharmaceutical aerosol formulation can be a solution, suspension, emulsion, semisolid, or powder. Topical foam products usually have an emulsion product concentrate. The propellant provides the force that expels the product concentrate from the container and additionally is responsible for the delivery of the formulation as a foam. The propellant can also serve as a solvent for the pharmaceutical actives or functional excipients that make up the product concentrate.

Unfortunately, the formulation of stable aerosol foams containing APIs with low water solubility can be difficult. APIs with low water solubility may not be consistently delivered in sufficiently high concentrations to produce the desired therapeutic effect after long term or accelerated storage conditions and/or the bioavailability could be reduced. In addition, undissolved APIs can clog the valve. This is more likely when a low water soluble API is used in a formulation which contains significant amounts of water such as an oil in water emulsion. A stable foam product containing completely dissolved APIs with low water solubility would improve efficacy and patient compliance.

### Propellants

A propellant is used to create pressure within a container and expel a product concentrate from the container. Propellants are chemicals with a vapor pressure greater than atmospheric pressure at 40° C. (105° F.). Pharmaceutical aerosols are commonly made using propellants such as chlorofluorocarbons, fluorocarbons (trichloromonofluoromethane, dichlorodifluoromethane), hydrocarbons (propane, butane, isobutane), hydrochlorofluorocarbons and hydrofluorocarbons, and compressed gases (nitrogen, NO₂, CO₂).

Chlorofluorocarbon (CFCs) propellants have been used for many years, however, due to their role in depleting the ozone layer, the use of CFCs has been significantly reduced.

Hydrochlorofluorocarbons (HCFCs) and hydrofluorocarbons (HFCs) differ from CFCs in that they may or may not contain chlorine and have one or more hydrogen atoms. HCFCs and HFCs have a lower impact on the ozone layer as they break down in the atmosphere at a faster rate than the CFCs. HCFCs and HFCs are used in topical pharmaceuticals. HCFCs and HFCs have a greater miscibility with water and therefore are more useful as solvents compared to the other propellants. For foam concentrates that consist of oil-in-water emulsions, HCFCs and HFCs readily blend with the continuous phase of the emulsion and provide excellent topical drug delivery vehicles for highly water-soluble actives, such as urea and salicylic acid. KERAFOAM® 42 Emollient Foam is a keratolytic emollient foam which is a tissue softener for skin and/or nails that contains urea, preservatives, buffering agents, water, ceteareth-10 phosphate, cetearyl alcohol and dicetyl phosphate. SALKERA® Emollient Foam is a keratolytic that contains 6% salicylic acid USP incorporated into an aqueous based emollient foam vehicle that contains moisturizers, preservatives, buffering agents, water, ceteareth-10 phosphate, ceteth-20 phosphate, cetostearyl alcohol, dicetyl phosphate and propylene glycol.

Hydrocarbon (HCs) propellants are used in topical pharmaceutical aerosols because of their lower environmental impact, their low toxicity and their nonreactivity. HCs are also useful in making three phase (two layer) aerosols because their density is less than 1 and they are immiscible with water. The hydrocarbons remain on top of the aqueous layer and provide the force to push the contents out of the container. They contain no halogens and therefore hydrolysis does not occur making these good propellants for water based aerosols. Unfortunately, hydrocarbon propellants are flammable and can explode. The flammability can be reduced by mixing the hydrocarbons with other liquefied gases. The liquid hydrocarbon propellants inside the canister can poorly mix with the internal oil phase of the oil-in-water emulsion and destabilize the foam concentrate. This results in a lack of content uniformity for the emitted doses from the canister. For this reason, an oil-in-water emulsion aerosol foam product incapable of depleting the ozone layer has not been developed that contains a water insoluble API.

TABLE 1
Properties of Hydrocarbon Propellants

| | | | | | Liquid |
|---|---|---|---|---|---|
| | | | V.P. | B.P. | Density |
| | | | @70° F. | ° F. | @68° F. |
| Name | Formula | No. | (psia) | (1 atm) | (g/mL) |
| Propane | C₃H₈ | A-108 | 124.7 | −43.7 | 0.50 |
| Isobutane | C₄H₁₀ | A-31 | 45.1 | 10.9 | 0.56 |
| Butane | C₄H₁₀ | A-17 | 31.2 | 31.1 | 0.58 |

Propane, butane, and isobutane are the most commonly used hydrocarbons. They are used alone or as mixtures to obtain the desired vapor pressure, density, and degree of flammability. Blends of propane, iso-butane and n-butane are usually designated as “AP” or “NIP” followed by a dash and number that is the pounds per square inch pressure (as determined with a pressure gauge) for the particular propellant blend at 70° F. For example, the AP-48 propellant is a 31:23:46 Propane:Isobutane:Butane blend that results in 48 psig in the can at 70° F. while the AP-70 propellant is a 55:15:30 Propane:Isobutane:Butane blend that results in 70 psig in the can at 70° F.

Inert and compressed-gas propellants expel the product concentrate in essentially the same form as it was placed into the container. The pressure of the compressed gas is in the headspace of the aerosol container. Compressed gas propellants are readily available, cheap and nonflammable, however, the pressure in the can is reduced as the product is used up. For pharmaceutical products this steady decrease in pressure with each actuation can result in the first dose of active delivered being significantly different than the last dose of active delivered from the canister. Also, once the compressed gas is depleted, any remaining product in the canister cannot be administered to the patient. For these reasons compressed-gas propellants are typically not used for pharmaceutical aerosols.

### Product Concentrates

An aerosol foam is produced when an oil in water emulsion product concentrate is mixed with a propellant and the propellant is in the internal oil phase of the emulsion. If the propellant is in the external phase (i.e., like a water in oil emulsion), foams are not created but sprays or wet streams result. A quick breaking foam creates a foam when emitted from the container but the foam collapses in a relatively short time. This type of foam is used to apply the product concentrate to a large area without having to manually rub or spread the product. The active drug is more rapidly available because the foam quickly collapses. Stable foams are produced when surfactants are used that have limited solubility in both the organic and aqueous phases. Surfactants concentrate at the interface between the propellant/oil phase and the aqueous phase to form a thin film referred to as the “lamella.” It is the specific composition of this lamella that dictates the structural strength and general characteristics of the foam. Thick and tightly layered lamellae produce very structured foams which are capable of supporting their own weight.

The emulsifier or surfactant used to formulate the product concentrate and the use of alcohol in the formulation are two of the most important components in a topical pharmaceutical foam. Surfactants in emulsion aerosols can include fatty acids saponified with triethanolamine, anionic surfactants, and more recently nonionic surfactants such as the polyoxyethylene fatty esters, polyoxyethylene sorbitan esters, alkyl phenoxy ethanols, and alkanolamides. The first dermatological foams contained high levels of alcohol (˜60% ethanol) and used the nonionic surfactant Polysorbate 60 and hydrocarbon propellants to create a quick breaking foam. The topical foams Olux® (clobetasol), Luxiq® (betamethacone), Lexette® (halobetasol) and Evoclin® (clindamycin) are high alcohol foams. Unfortunately, high alcohol foams were found to sting and burn for some psoriasis patients, and alcohol was removed from the clobetasol foam and Polysorbate 60 was replaced with polyoxyl 20 cetostearyl ether to launch the first emollient topical pharmaceutical foam Olux-E®. Finacea® topical foam has a very similar composition to Olux-E® since it contains Propylene glycol but no alcohol and uses the surfactant blend of polysorbate 80 and polyoxyl 40 stearate to form the foam lamella. The latest advance in topical pharmaceutical foam technology is Amzeeq® topical minocycline foam for the treatment of acne and rosacea. This product does not contain solvents but uses a blend of multiple natural oils to dissolve minocycline combined with hydrogenated castor oil as the surfactant to form foam lamella.

### BRIEF SUMMARY OF THE INVENTION

The present invention is directed to an aerosol foam composition comprising an API with water solubility below 60 mg/l. The aerosol foam composition is preferably an oil in water emulsion containing an emulsifier blend of cetearyl alcohol, dicetyl phosphate, and ceteareth-10 phosphate; in combination with a propellant. The propellant is a mixture of liquefied hydrocarbon gases preferably a propane/isobutane/butane blend. The hydrocarbon propellant results in an aerosol foam containing an API with low water solubility which is stable, has consistent physical properties, excellent aesthetics, and no discernable degradation of the API after long term (storage at ambient temperature for more than 24 months) or accelerated storage conditions (storage at 40° C. and 75% relative humidity for 6 months).

### BRIEF DESCRIPTION OF THE DRAWINGS

FIGS. 1A-1E show acceptable and unacceptable foams. FIGS. 1A-1D show acceptable foam structures including an expanding foam, a quick breaking foam, a stiff foam and a stout foam immediately after dispensing and 5 minutes after dispensing.

FIG. 1E shows an unacceptable foam with inadequate mixing of the propellant and the concentrate, resulting in sputtering during dispensing.

FIG. 2 shows acceptable foams containing ketoconazole according to formulations 5-8, 5 minutes after dispensing. Formulations 5 and 7 produced acceptable expanding foams. Formulation 6 produced an acceptable stout foam. Formulation 8 produced an acceptable stiff foam.

FIG. 3 shows acceptable and unacceptable foams containing econazole nitrate according to formulas 9-12, 5 minutes after dispensing. Formulation 9 produced an unacceptable sputter foam. Formulations 10 and 12 produced acceptable stout foams. Formulation 11 produced an unacceptable sputter foam.

FIG. 4 shows acceptable foams containing ivermectin according to formulas 13-16, 5 minutes after dispensing. Formulations 13-15 produced acceptable expanding foams and formulation 16 produced an acceptable quick breaking foam.

FIG. 5 shows acceptable quick breaking foams containing clobetasol propionate according to formulations 17-20, 5 minutes after dispensing.

FIG. 6 shows acceptable and unacceptable foams containing oxymetazoline according to formulations 21-24, 5 minutes after dispensing. Formulation 21 produced an acceptable stout foam. Formulation 22 produced an unacceptable sputter foam. Formulations 23 and 24 produced acceptable stiff foams.

### DETAILED DESCRIPTION OF THE INVENTION

Topical application of potent pharmacological agents for treating skin diseases has been found to provide superior delivery, lower systemic exposure and greater ease of use for patients. The molecular structure of the compound ultimately dictates the ability of the drug to cross the epithelium of the tissue to which the product is applied. For cutaneous application, selection of the components of the formulation dictates the maximum skin permeation that the formulator can achieve. The present invention is suitable for use with many APIs, particularly APIs with low solubility in water. Examples of low water soluble APIs suitable for use with the present invention include but are not limited to ketoconazole, econazole nitrate, ivermectin, clobetasol propionate, calcipotriene, halobetasol propionate, tazarotene, oxymetazoline free base and desonide. An API with low water solubility is hereby defined as an API with a water solubility of 60 mg/l or less. Creams, lotions, gels, ointments, aerosol foams and solutions are just a few of the more familiar forms of topical formulations that often contain completely dissolved APIs for application to the skin as disclosed regarding roflumilast in U.S. Pat. No. 5,712,298 (the “'298 patent”), incorporated herein by reference (col 12, lines 37-64). For treatment of such dermatoses, emulsions, suspensions, gels or solutions for topical application have been described, although the low solubility of the compound has limited those applications.

A cream formulation, containing an API with low water solubility was combined with a propellant. The foam concentrate was formulated to produce a foam which does not collapse after discharge from the container; has low shear sensitivity so that only minimal rubbing is required; is non-irritating, non-allergenic, and non-toxic; and keeps the API solubilized. Additionally, the aerosol foam vehicle contains a propellant that has minimal or no impact on the ozone layer of the atmosphere. The components in the foam concentrate and the propellants can be adjusted to produce foams with different properties such as expanding foams, quick breaking foams, stiff foams and stout foams. Preferably, the product expressed from the canister is a smooth white or off-white foam having uniform bubbles which are able to support their own weight until initiation of rub-in. As soon as rub-in is initiated the foam quickly breaks to evenly spread across the application site. The product preferably has a foam half-life of more than 60 seconds. The amount of the foam dispensed by the canister may or may not be metered to dispense a consistent amount of the foam and a consistent dosage of the API.

The aerosol foam includes 1-10%, preferably 2-5%, of an emulsifier containing an alkyl phosphate anionic surfactant or blend of alkyl phosphate surfactants to ensure mixing with the propellant. Emollients or oils are included in amounts which produce an aesthetically pleasing foam. Preferably, the emollients include 2-6%, preferably 5%, petrolatum; and 2-3%, preferably 2.5%, isopropyl palmitate. Preferably the oils include 8-12% diisopropyl adipate and 8-12% oleyl alcohol.

The propellant provides the force that expels the product concentrate from the container and additionally is responsible for the delivery of the formulation as a foam. Since the aerosol foam propellant is a mixture of liquefied hydrocarbon gases, it can also serve as a solvent for the API or can be mixed with the internal oil phase of the emulsion of the product concentrate. The use of a hydrocarbon propellant may reduce or eliminate the need for additional solvents such as hexylene glycol and DEGEE (diethylene glycol monoethyl ether). Hexylene glycol is preferably in an amount of 0-20% w/w and DEGEE is preferably in an amount of 10-35% w/w. The hydrocarbon propellant partially mixes with the API concentrate, but primarily forms a separate liquid layer (lower density than the concentrate) inside the can. This is commonly referred to as a three-phase pharmaceutical aerosol. Thus, it is necessary to shake the can to evenly distribute the propellant throughout the finished product prior to applying the emitted foam to the skin of the patient.

### Product Concentrate

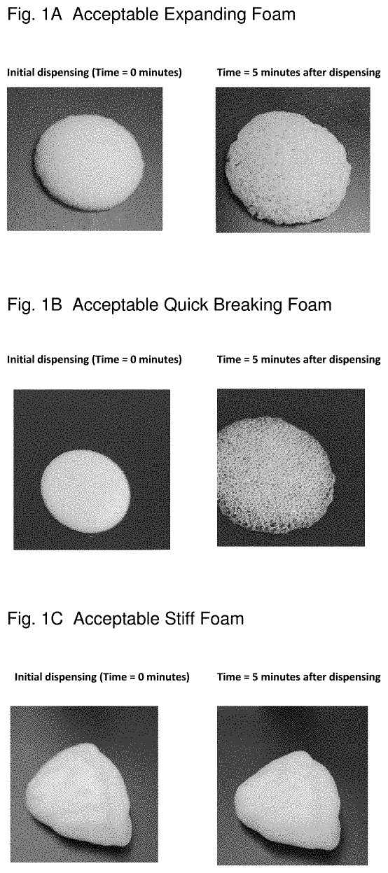

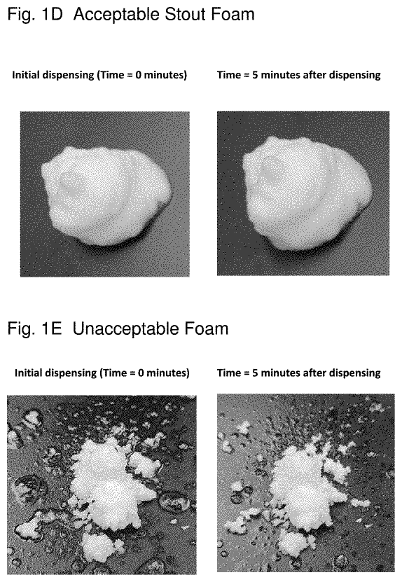

The product concentrate in the foam consists of an oil-in-water emulsion of the low water soluble active ingredient, 10-35% diethylene glycol monoethyl ether, NF (TRANSCUTOL®P), 30-80% water, 7.5-20% oil phase, and 1-10%, in some embodiments 2-5% of the anionic surfactant based emulsifying wax Crodafos CES™. These components produce a foam for treatment of the scalp and face. This foam concentrate can form a quick breaking, expanding foam which collapses in a relatively short time after application (without rub-in) to the skin. This type of foam is used to apply the product concentrate to a large area without having to manually rub or spread the product. The active drug is more rapidly available because the foam quickly collapses and foams are more easily applied to skin areas having a high density of terminal hairs, i.e. the scalp. Depending on the low water soluble active and the amount of oil phase and/or emulsifying wax selected, this foam concentrate can also form a stable foam that does not break until the product emitted from the canister undergoes rub-in. The viscosity values for a range of Crodafos CES™ concentrations is given in Table 3. The 10% Crodafos CES™ containing 0.3% roflumilast as the low water soluble active produced an unacceptable foam that “sputtered” when emitted from the can. Sputter (represented in FIG. 1E) indicates inadequate mixing between the liquid propellant and emulsion foam concentrate inside the canister. However, the 2% Crodafos CES™ formulation with 0.3% roflumilast produced an acceptable stiff foam (represented in FIG. 1C).

The final composition of a 0.3% roflumilast foam is given in Table 2. The roflumilast emitted foam product having this composition has consistent physical properties, excellent aesthetics, no discernable roflumilast degradation after long term (storage under ambient conditions for 24 or more months) or accelerated storage conditions (storage at 40° C. and 75% relative humidity for 6 months) and during development showed acceptable but variable roflumilast assay results. A series of quality by design experiments focused on the analytical method of sample preparation, optimization of the product concentrate and characterization of packaging compatibility were completed. It was determined that variability in assay results could be minimized by including a hexane extraction during sample preparation.

TABLE 2
Composition of ARQ-154 foar product.

| | Concentration in ARQ-154 |
|---|---|
| Ingredient | Concentrate |
| Roflumilast | n/a for vehicle or 0.3% |
| DEGEE (Transcutol P) | 25% w/w |
| Petrolatum | 5.0% w/w |
| Isopropyl Palmitate | 2.5% w/w |
| CRODAFOS ™ CES | 2, 4, 6, 8 or |
| cetearyl alcohol | 10% w/w |
| dicetyl phosphate | |
| ceteareth-10 phosphate | |
| Hexylene Glycol | 2% w/w |
| Methylparaben | 0.2% |
| Propylparaben | 0.05% |
| Purified Water | q.s. ad 100% |
| Propane/Isobutane/Butane Blend (AP-70 | NA* |
| or AP-48) | |

*8-10 grams of propellant is added to 64 grams (target) of the emulsion concentrate to deliver a minimum 60 grams of foam product

Preferred viscosities are between 4000-11,000 centipoise (cP). The viscosity was tested using a Brookfield Viscometer which determines the viscosity by measuring the force to turn the spindle in the sample at a given rate. A regular viscosity spring (RV) was used with a #14 spindle at 30 rpm, sample chamber 6R. However, any digital viscometer (DVE, DV1, DV2, or DV3) is suitable for determining viscosity. The time to read was 2 minutes and the temperature was controlled room temperature (CRT, 20-25° C.).

TABLE 3
Viscosity values for varying levels of Crodafos CES in the 2% Crodafos
CES ™ formulation of 0.3% roflumilast formulation shown in Table 2

| | % Crodafos | Viscosity | |
|---|---|---|---|
| Sample | CES | (cP) | Appearance |
| 2017-014-95-18 | 10 | 29130 | Smooth, Thick White Cream |
| 2107-014-95-38A | 8 | 10750 | Smooth, White Cream |
| 2107-014-95-38B | 6 | 9290 | Smooth, White Cream |
| 2107-014-95-38C | 4 | 6330 | Smooth, White Cream |
| 2107-014-95-38D | 2 | 4190 | Smooth, Thin White Cream |

The emitted foam product containing a low water soluble active has consistent physical properties, excellent aesthetics, acceptable assay results and no harmful quantities of degradation products after long term (storage under ambient conditions for 3 or more months) or accelerated storage conditions (storage at 40° C. and 75% relative humidity for 3 to 6 months). Typical data generated for a low water soluble active is given in Table 4 (long-term stability storage) and Table 5 (accelerated stability storage) for the 2% Crodafos CES™ formulation of 0.3% roflumilast formulation described in Table 2. The preferred aesthetics of the foam concentrate was optimized by reducing the emollients by half (5% rather than 10% for petrolatum and 2.5% rather than 5.0% for isopropyl palmitate). Only two 2% Crodafos CES™ foam concentrate formulations were compared to optimize the aesthetics of the foam formulation. The foam concentrate having 15% combined moisturizers felt “oily” during rub-in compared to the foamed concentrate containing 7.5% combined moisturizers. Since the foam product was formulated to treat the scalp and facial seborrheic dermatitis skin (both anatomical sites known to have oily skin prior to foam application), it was considered an aesthetic advantage to reduce the moisturizer content of the foam compared to the cream. To compensate for the removal of 15.5% of the emulsifier/emollients, the amount of water in the foam is increased to just over 65% in the foam concentrate compared to ˜50% water in the roflumilast cream. Three months of informal stability data for 64 grams product concentrate formulation (Table 2) gassed with 8 grams of AP-70 propellant is shown in Tables 4 and 5.

TABLE 4
Stability Data for the 2% Crodafos CES ™ formulation of
0.3% roflumilast formulation stored at 25° C. Inverted

| Test | T = 0 | | T = 1 MO | | T = 2 MO | | T = 3 MO | |
|---|---|---|---|---|---|---|---|---|
| Description* | Meets | | Meets | | Meets | | Meets | |
| pH | 5.44 | | 5.26 | | 5.29 | | 5.29 | |
| Pressure @ 25° C. | 58 | psi | 75 | psi | 65 | psi | 73 | psi |
| Delivery Rate | 1.64 | g/sec | 2.18 | g/sec | 2.38 | g/sec | 1.91 | g/sec |
| @ 25° C. ** | | | | | | | | |
| Foam Density | 0.091 | g/mL | 0.112 | g/mL | 0.104 | g/mL | 0.104 | g/mL |

| | | | | |
|---|---|---|---|---|
| \hat{ }Assay roflumilast | 99.0% | 99.9% | 97.0% | 99.4% |
| \hat{ }Assay methylparaben | 99.7% | 99.6% | 100.2% | 98.1% |
| \hat{ }Assay propylparaben | 99.7% | 99.3% | 99.5% | 98.4% |

\hat{ }Assay % label claim results are the average value of n = 9 replicates for each test and timepoint, normalized against the bulk formulation concentrate.
*Description: White, opaque, foam with small, compact bubbles. Foam is not runny.
** (Average Delivery Rate grams/second): Method: USP 603

TABLE 5
Stability Data for the 2% Crodafos CES ™ formulation of 0.3% roflumilast
formulation stored at 40° C. Inverted

| Test | T = 0 | | T = 1 MO | | T = 2 MO | | T = 3 MO | | T = 6 MO |
|---|---|---|---|---|---|---|---|---|---|
| Description* | Meets | | Meets | | Meets | | Meets | | Meets |
| pH | 5.44 | | 5.31 | | 5.34 | | 5.38 | | 5.28 |
| Pressure @ 25° C. | 58 | psi | 70 | psi | 65 | psi | 70 | psi | NT |
| Delivery Rate @ | 1.64 | g/sec | 2.20 | g/sec | 2.28 | g/sec | 1.64 | g/sec | NT |
| 25° C. ** | | | | | | | | | |
| Foam Density | 0.091 | g/mL | 0.110 | g/mL | 0.100 | g/mL | 0.092 | g/mL | NT |

| | | | | | |
|---|---|---|---|---|---|
| \hat{ }Assay roflumilast | 99.0% | 96.1% | 93.4% | 94.8% | 98.1% |
| \hat{ }Assay methylparaben | 99.7% | 99.3% | 99.5% | 97.3% | 96.1% |
| \hat{ }Assay propylparaben | 99.7% | 98.8% | 98.9% | 97.8% | 97.1% |

NT = Not Tested
\hat{ }Assay % label claim results are the average value of n = 9 replicates for each test and
timepoint, normalized against the bulk formulation concentrate.
*Description: White, opaque, foam with small, compact bubbles. Foam is not runny.
** (Average Delivery Rate grams/second): Method: USP 603

### Propellants

A hydrocarbon propellant has been found to result in a topical foam with the desired properties. They contain no halogens and therefore hydrolysis does not occur making these good propellants for water-based aerosols such as an oil in water emulsion comprising low water soluble APIs.

Six different hydrocarbon propellants, one N-Butane/dimethyl ether blend and one hydrofluorocarbon propellant were screened with the 2% Crodafos CES™ formulation of 0.3% roflumilast formulation described in Table 2. The six hydrocarbon propellants were Isobutane (A-31), N-Butane (A-17), Propane/Isobutane (A-46), Propane/Isobutane (A-70), Propane/Isobutane/N-Butane (AP-70), Aeropin 35 (Aeropin 35 is a blend of Propane/Isobutane/N-Butane having a vapor pressure of 35 psig at 70° F. such that the ratio of Isobutane to N-Butane is fixed at 2/3) and Butane 48 (Butane 48 is a 30.8/22.9/45.8/0.5 ratio of Propane/Isobutane/N-Butane/Isopentane). The hydrocarbon blend with dimethyl ether (DME) was 53% DME and 47% N-Butane. The hydrofluorocarbon propellant was 1,1,1,2-tetrafluoroethane (HFA 134a). The AP-70 propellant produced the best quality foam in the initial foam propellant screening study. HFA-134a (1,1,1,2-tetrafluoroethane), the propellant used in highly water-soluble urea (KERAFOAM® 42) and salicylic acid (SALKERA®) emollient foams, was combined with Formulation 1. The emitted product was a clumpy, gelatinous looking material that did not comprise gas bubbles distributed in a liquid. Table 1 provides the properties of the three hydrocarbon propellants that are blended to create the “AP” or “NIP” designated aerosol propellants and Table 6 lists the appearance of aerosolize topical foam products.

TABLE 6
Foam appearance for the 2% Crodafos CES ™ formulation
containing 0.3% roflumilast combined
with different commercially available propellants

| Propellant | Tradename | Foam Appearance |
|---|---|---|
| N-Butane | Aeropres | This propellant resulted in a runny looking |
| | A-17 | product upon dispensing that did not meet |
| | | appearance requirements for a foam. |
| Propane/ | Aeropres | This propellant resulted in a runny looking |
| Isobutane | A-46 | product upon dispensing that did not meet |
| | | appearance requirements for a foam. |
| Propane/ | Aeropres | This propellant resulted in an acceptable stiff |
| Isobutane | A-70 | foam (see FIG. 1C) with bubbles that were |
| | | very small and uniform in size. Foam looked |
| | | good and bubbles seemed to remain very |
| | | small after several minutes. Can filled with |
| | | 64.2 g bulk and 4.9 g propellant. Pressure: |
| | | 75 psi |
| Propane/ | Aeropres | This propellant resulted in an acceptable, |
| Isobutane/ | AP-70 | smooth, white, stiff foam product (see FIG. |
| N-Butane | | 1C) having bubbles that were small and |
| | | uniform in size. The foam supported its own |
| | | weight upon dispensing but readily broke |
| | | during rub-in. |
| Propane/ | Aeropres | This propellant resulted in an acceptable, |
| Isobutane/ | AP-48 | smooth, white, stiff foam product (see FIG. |
| N-Butane | | 1C) having bubbles that were small and |
| | | uniform in size. The foam supported its own |
| | | weight upon dispensing but readily broke |
| | | during rub-in. |
| blend of | Aeropin 35 | This propellant resulted in bubbles that were |
| Propane/ | AP-35 | very small and uniform in size. An |
| Isobutane/ | | acceptable stiff foam (see FIG. 1C) was |
| N-Butane | | produced. No sputtering was observed. Can |
| having a | | filled with 62.6 g bulk and 5.5 g propellant. |
| vapor | | Pressure: 56 psi |
| pressure | | |
| of 35 | | |
| psig at 70° F. | | |
| such as | | |
| the ration of | | |
| Isobutane to | | |
| N-Butane is | | |
| fixed at 2/3 | | |
| 53% | n/a | Runny looking product upon dispensing that |
| Dimethyl | | did not meet appearance requirements for a |
| Ether | | foam. |
| (DME) and | | |
| 47% N- | | |
| Butane | | |
| 1,1,1,2- | HFA 134a | Propellant did not mix well with the product |
| tetra- | | and produced a clumpy, gelatinous looking |
| fluoroethane | | product when dispensed. Did not meet |
| | | appearance requirements for a foam. |

The aesthetics of the foam formulation shown in Table 2 (64 grams concentrate that contained 2% Crodafos CES™ formulation of 0.3% roflumilast) when gassed with 8 grams of either AP-48 or AP-70 propellant were compared. The AP-48 propellant is a 31:23:46 Propane:Isobutane:Butane blend while AP-70 propellant is a 55:15:30 blend of the same hydrocarbons. While both foams were found completely acceptable, the firmer appearance and slightly slower breaking of the AP-48 propellant foam was preferred by about two-thirds of the individuals testing the products. The other third of the testers had either no preference or a slight preference for the quicker breaking AP-70 foam. It was concluded that both the AP-48 and AP-70 hydrocarbon blends show good topical foam characteristics and excellent aesthetics. By adjusting the ratio of propane to the butanes, any pressure between 48 and 70 psig can be achieved. In terms of aesthetics, any ratio of the hydrocarbon propellant blend of Propane/Isobutane/N-Butane that gives a pressure around 48-70 psig at 70° F. has been shown acceptable.

### The Foam Product

An aerosol foam is produced when the oil in water emulsion product concentrate is mixed with the liquid hydrocarbon propellant and the propellant is in the internal oil phase. If the propellant is in the external phase (i.e., like a water-in-oil emulsion), foams are not created but sprays or wet streams result. Stable foams are produced when surfactants are used that have limited solubility in both the internal oil and external aqueous phases. Surfactants concentrate at the interface between the propellant/oil phase and the aqueous phase to form a thin film referred to as the “lamella.” It is the specific composition of this lamella that dictates the structural strength and general characteristics of the foam. Thick and tightly layered lamellae produce very structured foams which can support their own weight. In a preferred embodiment, two alkyl phosphate surfactants are used which are not commonly used in a topical foam product. These alkyl phosphate surfactants are in the emulsifier Crodafos CES™.

For all topical pharmaceutical foams, it is assumed that all propellant is released from the formulation when the last lamella ruptures (foam bubble breaks). The specific composition of the foam lamella dictates the structural strength and general characteristics of the foam. The liquid crystal stabilized oil-in-water emulsion low water soluble API concentrate has multiple Crodafos CES lamella surrounding each oil droplet. The solvent DEGEE (diethylene glycol monoethyl ether) is both water and oil miscible, thus it is likely partitioned between the oil and water phases and distributed within the multiple lamella at the interface of the emulsion. The concentrate is added to the can, the valve crimped into place on the top of the can and the propellant added under pressure through the valve of the primary container closure system. Within the can, some of the liquid propellant partitions into the oil phase. When the can is shaken, the propellant readily mixes with the oil droplets of the concentrate to form a milky white, emulsion in the can. As the propellant transitions from a liquid under pressure to a gas when emitted from the can, the volume of liquid propellant resident within the oil globule rapidly expands to become the hydrocarbon gas bubble trapped within the lamella of the foam. As the propellant expands, the multiple lamella of the droplet quickly becomes the single lamella of the foam. Once the pressure associated with the volume of gaseous propellant exceeds the strength of the surfactant lamella, the foam cell breaks and API concentrate drains to the surface of the skin.

Different hydrocarbon blends can be used in the propellant to change the properties of the foam. For example, the AP-70 propellant contains more propane to produce a higher-pressure propellant bubble, and thus should make slightly larger foam bubbles. The AP-70 propellant should also cause the foam bubbles to expand somewhat after the foam comes out of the can and be a little “faster breaking” than a foam having the lower pressure AP-48 as the propellant. The firmer appearance and slightly slower breaking of the AP-48 propellant foam was preferred in a side-by-side comparison of vehicle foams gassed with either the AP-48 or AP-70 propellants. Both the AP-48 and AP-70 hydrocarbon blends show good topical foam characteristics and excellent aesthetics.

Compositions according to the present invention may be formulated with additional components such as fillers, carriers and excipients conventionally found in cosmetic and pharmaceutical topical products. Additional components including but not limited to preservatives (e.g. p-hydroxybenzoic esters, benzyl alcohol, phenylmercury salts, chlorocresol), antioxidants, sequestering agents, stabilizers, buffers, pH adjusting solutions, skin penetration enhancers, film formers, dyes, pigments, diluents, bulking agents, fragrances and other excipients to improve the stability or aesthetics, may be added to the composition.

The low water soluble active pharmaceutical ingredient can be selected from the group of actives with water solubility below 60 mg/liter. This group of actives includes but is not limited to ketoconazole, econazole nitrate, ivermectin, clobetasol propionate, calcipotriene, halobetasol propionate, tazarotene, oxymetazoline free base and desonide.

TABLE 7
Water Solubility Data:

| Active Pharmaceutical Ingredient | Water Solubility (mg/liter) |
|---|---|
| ketoconazole | 0.09 |
| roflumilast | 0.54 |
| econazole nitrate | 1.48* |
| ivermectin | 4 |
| clobetasol propionate | 4 |
| calcipotriene | 13.5 |
| halobetasol propionate | 14 |
| tazarotene | 32 |
| oxymetazoline free base | 52* |
| desonide | 59 |
| azelaic acid | 2,140 |
| minoxidil | 2,200 |
| salicylic acid | 2,480 |
| minocycline hydrochloride | 50,000 |
| oxymetazoline hydrochloride | 145,000 |
| clindamycin phosphate | 250,000 |
| urea | 1,190,000 |

*ALOGPS calculated value

Compositions according to the present invention may be formulated with active agents in addition to the low water soluble active pharmaceutical ingredient depending on the condition being treated. The additional active agents include but are not limited to Anthralin (dithranol), Azathioprine, Tacrolimus, Coal tar, Methotrexate, Methoxsalen, Salicylic acid, Ammonium lactate, Urea, Hydroxyurea, 5-fluorouracil, Propylthiouracil, 6-thioguanine, Sulfasalazine, Mycophenolate mofetil, Fumaric acid esters, Corticosteroids (e.g. Aclometasone, Amcinonide, Betamethasone, Clobetasol, Clocotolone, Mometasone, Triamcinolone, Fluocinolone, Fluocinonide, Flurandrenolide, Diflorasone, Desonide, Desoximetasone, Dexamethasone, Halcinonide, Halobetasol, Hydrocortisone, Methylprednisolone, Prednicarbate, Prednisone), Corticotropin, Vitamin D analogues (e.g. calcipotriene, calcitriol), Acitretin, Tazarotene, Cyclosporine, Resorcinol, Colchicine, Adalimumab, Ustekinumab, Infliximab, bronchodialators (e.g. beta-agonists, anticholinergics, theophylline), and antibiotics (e.g. erythromycin, ciprofloxacin, metronidazole).

The pharmaceutically active agent which has low solubility in water can be encapsulated to control the release rate from the composition and to protect the active agent from degradation. Encapsulation can also be used to modify skin penetration. Methods for encapsulating active pharmaceutical ingredients are known in the art and include but are not limited to encapsulation in liposomes, microparticles, nanoparticles, nanocarriers, nanospheres, microspheres, microcapsules, nanocapsules, nanosponges, and microsponges.

The foam composition can be administered on a schedule appropriate for the condition being treated, preferably the foam composition is administered one or more times per day, more preferably the composition is administered 1-2 times per day.

The composition can be used in veterinary and in human medicine for the treatment and prevention of all diseases regarded as treatable or preventable by using a low water soluble active, including but not limited to proliferative, inflammatory and allergic dermatoses such as psoriasis (vulgaris), eczema atopic dermatitis; parasitic infestations; fungal skin infections; bacterial or fungal overgrowth; acne; rosacea and erythematotelangiectatic rosacea; and lichen sclerosus.

The following examples are provided to enable those of ordinary skill in the art to make and use the methods and compositions of the invention. These examples are not intended to limit the scope of what the inventor regards as the invention. Additional advantages and modifications will be readily apparent to those skilled in the art.

### EXAMPLES

### Example 1

Eight different hydrocarbon propellants, a 47/53 wt/wt blend of N-Butane/dimethyl ether and the hydrofluorocarbon HFA 134a were added to the foam concentrate [either Formulation 1 or Formulation 2] listed in Table 8 and the emitted foam appearance was noted after gentle shaking of the canister. Target proportions were 5 grams propellant added to 62 grams foam concentrate. As seen in Table 6, the use of either N-Butane or Isobutane alone as a propellant and blends of propane and isobutane produced a runny product that did not meet appearance requirements for a foam. However, a Propane/Isobutane/N-Butane blended propellant produced an emitted foam that was smooth, white and uniform. This foam using the three-hydrocarbon propellant blend initially supported its own weight but readily broke during rub-in. The addition of isopentane to the Propane/Isobutane/N-Butane propellant blend destabilized the emitted foam and produced a runny looking product.

TABLE 8

| Foam Concentrate | | |
|---|---|---|
| Composition | Formulation 1 | Formulation 2 |
| Roflumilast | 0.3 or 0.15 | 0.3 or 0.15 |
| Petrolatum, USP | 5.0 | 10.0 |
| Isopropyl Palmitate, NF | 2.5 | 5.0 |
| Crodafos CES | 2.0 | 2.0 |
| cetostearyl alcohol | NMT 1.6 | NMT 1.6 |
| dicetyl phosphate | NMT 0.4 | NMT 0.4 |
| ceteareth-10 phosphate | NMT 0.4 | NMT 0.4 |
| Diethylene Glycol | 25.0 | 25.0 |
| Monoethyl Ether, NF | | |
| (Transcutol P) | | |
| Hexylene Glycol, NF | 2.0 | 2.0 |
| Methylparaben, NF | 0.20 | 0.20 |
| Propylparaben, NF | 0.050 | 0.050 |
| 1N NaOH, NF | q.s. ad pH 5.5 | q.s. ad pH 5.5 |
| Purified Water, USP | q.s. ad 100% | q.s. ad 100% |

Dimethyl ether is commonly added to a hydrocarbon propellant to increase solubility in the canister of water-insoluble actives, especially if the foam concentrate contains alcohol (ethanol or isopropyl alcohol). As seen in Table 6, the addition of dimethyl ether to N-butane resulted in a runny looking product upon dispensing that did not meet appearance requirements for a foam.

HFA-134a (1,1,1,2-tetrafluoroethane), the propellant used in highly water-soluble urea (KERAFOAM®42) and salicylic acid (SALKERAG) emollient foams, was combined with Formulation 1. The emitted product was a clumpy, gelatinous looking material that did not comprise gas bubbles distributed in a liquid.

### Example 2 Determining the Dispersed Content Uniformity Throughout Canister Life

The appearance of 64 grams foam concentrate (Formulation 1 containing 0.15% roflumilast) when gassed with 5, 6, 8 or 10 grams of AP-70 propellant were compared. The emitted foam appearance for these four foam concentrate to propellant ratios was indistinguishable smooth, white foam products having gas bubbles that were small and uniform in size.

Additional analytical testing was completed on formulation 1 (containing 0.3% roflumilast) to determine dispersed roflumilast content uniformity throughout the canister life. Two clinically relevant doses (˜1 gram) were dispensed from the beginning of the can (initial actuations after ˜5 hand shakes of the can). The amount of foam dispensed was quantified by completing a difference weighing of the can and the assay results of the two separate foam extractions were averaged to give the “Beginning Average” value. 15 grams of foam was dispensed, and the canister was allowed to return to room temperature. An additional 5-6 hand shakes of the canister was followed by dispensing two clinically relevant doses (˜1 gram) from the middle of the canister. Assay results of the two separate foam extractions were averaged to give the “Middle Average” value. An additional 15 grams of foam was dispensed, the canister allowed to return to room temperature. This sequence of sampling was repeated to give the “End Average” data. Data comparing the “Beginning Average”, Middle Average” and “End Average” for lot PGX-C containing 10 grams of AP-70 propellant compared to a lot that contains 8 grams of AP-70 propellant is shown in Table 9.

According to USP<607> Pharmaceutical Foams-Product Quality Tests the dispersed content uniformity throughout canister life must not exceed 10%. This compendial method instructs to dispense quantities according to the labeled instructions separately collecting an appropriate amount of individually weighed foam drug product. The sample size should not exceed the maximum dose recommended by the product labeling for a single application. The labeled use instructions determine if the can should be shaken prior to expelling foam and the orientation (upright or inverted) when dispensing. Portions of foam should be retained corresponding to: 1) an initial portion from the filled canister, 2) a portion from the middle of the canister (in the range of 40%-60% of labeled canister content), and 3) the portion corresponding to the canister contents with 85% of the labeled contents delivered. The canister should be dispensed at room temperature. If the canister cools as a result of dispensing, the canister should be warmed to room temperature before subsequent delivery. Using an appropriate sample preparation (such as outgassing) and analytical method, determine the drug substance concentration in each of the three portions. None of the three results are outside of the product assay range. The maximum difference in the amount of active ingredient determined within the canister is NMT 10.0%, beginning, middle and end.

As seen in Table 9, the addition of 10 grams of HC propellant destabilizes the O/W emulsion in the canister. When the canister is shaken, the liquid propellant (specific gravity=0.54) mixes with the internal oil phase (petrolatum/isopropyl palmitate/cetostearyl alcohol−specific gravity=0.83) and causes the now swollen emulsion globules to rise (creaming of the emulsion) away from the inverted valve/actuator. Since the water insoluble active disproportionately resides surrounding the oil phase of the emulsion, repeating this process of shaking the canister and emitting the foam serves to concentrate active in the canister. When the O/W emulsion is destabilized to the point of exceeding the maximum difference limit (not more than 10%) specified for content uniformity throughout canister life according to USP<607>, the aerosol foam drug product is no longer commercially viable. For a target 64-gram fill of 0.3% roflumilast Formulation 1, increasing the amount of AP-70 hydrocarbon propellant, suddenly and unexpectantly destabilized the emulsion of the foam concentrate to make this foam drug product no longer acceptable for commercial pharmaceutical products.

TABLE 9

| Formulation 1 foam | | | | | |
|---|---|---|---|---|---|
| concentrate with | | Middle | End | | |
| a target fill | Beginning | Aver- | Aver- | % | Maximum |
| weight of 64 grams | Average | age | age | RSD | Difference |
| 10 grams HC | 93.3% | 94.5% | 105.4% | 6.8% | 12.1% |
| Propellant (EKG | | | | | |
| S190275) | | | | | |
| 8 grams HC | 92.6% | 94.8% | 98.2% | 3.0% | 5.6% |
| Propellant (EKG | | | | | |
| S200148) | | | | | |

HC Propellant is a blend of 31% Propane, 23% Isobutane and 46% n-butane

### Example 3 Effect of Increasing the Concentration of Diethylene Glycol Monoethyl Ether

Using the same USP<607> Pharmaceutical Foams—Product Quality Tests as detailed in Example 2 for determining the dispersed content uniformity throughout canister life, the effect of increasing the concentration of diethylene glycol monoethyl ether (Table 10) was determined.

TABLE 10

| Foam Concentrate | | | |
|---|---|---|---|
| Composition | Formulation 1 | Formulation 3 | Formulation 4 |
| Roflumilast | 0.3 | 0.3 | 0.3 |
| Petrolatum, USP | 5.0 | 5.0 | 5.0 |
| Isopropyl Palmitate, | 2.5 | 2.5 | 2.5 |
| NF | | | |
| Crodafos CES | 2.0 | 2.0 | 2.0 |
| cetostearyl alcohol | NMT 1.6 | NMT 1.6 | NMT 1.6 |
| dicetyl phosphate | NMT 0.4 | NMT 0.4 | NMT 0.4 |
| ceteareth-10 | NMT 0.4 | NMT 0.4 | NMT 0.4 |
| phosphate | | | |
| Diethylene Glycol | 25.0 | 35.0 | 40.0 |
| Monoethyl Ether, NF | | | |
| (Transcutol P) | | | |
| Hexylene Glycol, NF | 2.0 | 2.0 | 2.0 |
| Methylparaben, NF | 0.20 | 0.20 | 0.20 |
| Propylparaben, NF | 0.050 | 0.050 | 0.050 |
| 1N NaOH, NF | q.s. ad pH 5.5 | q.s. ad pH 5.5 | q.s. ad pH 5.5 |
| Purified Water, USP | q.s. ad 100% | q.s. ad 100% | q.s. ad 100% |

When the O/W emulsion is destabilized to the point of exceeding the maximum difference limit (not more than 10%) specified for content uniformity throughout canister life in USP<607>, the aerosol foam drug product is no longer commercially viable. For a target 64-gram fill of 0.3% roflumilast foam concentrate and 8-gram fill of AP-70 hydrocarbon propellant, the emulsion in the canister suddenly and unexpectantly destabilizes when the DEGEE concentration is increased from 35% to 40% (Table 10). The emulsion of this foam drug product containing 40% DEGEE is not acceptable for pharmaceutical commercialization.

TABLE 11

| Foam concentrate | | | | | Maxi- |
|---|---|---|---|---|---|
| (target fill weight of 64 | | Middle | End | | mum |
| grams) blended | Beginning | Aver- | Aver- | % | Differ- |
| with 8 grams of AP-70 | Average | age | age | RSD | ence |
| FORMULATION 1 | 92.6% | 94.8% | 98.2% | 3.0% | 5.6% |
| (EKG S190148) | | | | | |
| FORMULATION 3 | 97.2% | 99.6% | 100.7% | 1.8% | 3.2% |
| (EKG S200075) | | | | | |
| FORMULATION 4 | 96.4% | 99.0% | 131.3% | 17.9% | 34.9% |
| (EKG S200078) | | | | | |

### Example 4

As detailed in Example 2, two clinically relevant doses (˜1 gram) were dispensed from the beginning, middle and end of the can. The amount of foam dispensed was quantified by completing a difference weighing of the can and the assay results of the two separate foam extractions were averaged to give the beginning average (B), middle average (M) or end average (E) values shown in Table 11. After each pair of clinically relevant actuations, approximately 15 grams of foam was dispensed into a glass container, tightly closed, and stored for optional assay. These samples were labeled as the beginning retain (BR), middle retain (MR) and end retain (ER). The six assay values (which represents assay of the entire contents of the canister) for FORMULATION 4 from Table 10 is shown in Table 12.

TABLE 12

| | Begin- | Beginning | | Middle | | End |
|---|---|---|---|---|---|---|
| | ning | Retain | Middle | Retain | End | Retain |
| FORMU- | 96.4% | 69.4% | 99.0% | 72.2% | 131.3% | 111.0% |
| LATION 4 | | | | | | |

The data shown in Table 12 provides a dramatic example of how creaming of a foam concentrate emulsion within the canister can cause dramatic changes in dosing levels of active to the patient. From development of roflumilast emulsion formulations it is known that increasing the amount of DEGEE from 25% to 40% will increase the solubility of roflumilast in the foam concentrate, but increasing DEGEE above 35% also destabilizes the emulsion. The assay pattern after fully assaying the canister (Table 12) indicates that active is migrating to the portion of the emulsion containing roflumilast that is being retained in the canister during actuation. By walking through the assay steps, the data from Table 12 can be understood. The full can of product is shaken, and the beginning one-gram samples are dispensed with an assay value of 96.4%. The can is again shaken and approximately 15-grams of foam is dispensed into a jar in a single actuation—the roflumilast-rich, propellant swollen globules of the destabilized emulsion phase separate (creaming) and migrate away from the valve of the inverted canister. Creaming of the emulsion carries a disproportionate amount of roflumilast toward the interface between the emulsion and liquid propellant which assures that the “Beginning Retain” has a very low assay value of 69.4%. The can is allowed to return to room temperature, shaken and the short actuation, 1-gram middle samples are taken and assayed at 99.0% of label. Once again, due to the destabilized emulsion, roflumilast evades being dispensed from the canister during the long actuation during dispensing of the “Middle Retain” (72.2% of label). With about two-thirds of the three-phase pharmaceutical aerosol having been dispensed at low potency, the 1-gram end actuations have the highest assay value of 131.3% of label. The final long actuation to produce the “End Retain” assay value maintains the trend of having a lower roflumilast assay value (111.0% label) compared to end sample (131.3% label). Depending on how long the canister is held inverted after shaking, a physically unstable emulsion foam product could deliver 69% of the labeled dose or 131% of the labeled dose. Formulation 4 would not be a commercially viable pharmaceutical aerosol foam product.

### Example 5 Ratio of the Hydrocarbon Blend

The aesthetics of ARQ-154 foam formulation shown in Table 2 (64 grams concentrate) when gassed with 8 grams of either AP-48 or AP-70 propellant were compared. The AP-48 propellant is a 31:23:46 Propane:Isobutane:N-Butane blend while AP-70 propellant is a 55:15:30 blend of Propane:Isobutane:N-Butane. While both foams were found completely acceptable, the firmer appearance and slightly slower breaking of the AP-48 propellant foam was preferred by about two-thirds of the individuals testing the products. The other third of the testers had either no preference or a slight preference for the quicker breaking AP-70 foam. It was concluded that both the AP-48 and AP-70 hydrocarbon blends show good topical foam characteristics and excellent aesthetics. By adjusting the ratio of propane to the isobutane:N-Butane mixtures, any pressure between 48 and 70 psig can be achieved. In terms of aesthetics, any ratio of the hydrocarbon propellant blend of Propane/Isobutane/N-Butane that gives a pressure around 48-70 psig at 70° F. has been shown acceptable.

### Example 6. Formulations with Various Low Water Soluble APIs

TABLE 13

| | Concentration in Product Concentrate (% w/w) | | | |
|---|---|---|---|---|
| | For- | For- | For- | For- |
| | mulation | mulation | mulation | mulation |
| Ingredient | 5 | 6 | 7 | 8 |
| Ketoconazole | 2% | 2% | 2% | 2% |
| DEGEE (Transcutol P) | 25 | 15 | 20 | 10 |
| Petrolatum | 5.0 | 5.0 | — | — |
| Isopropyl Palmitate | 2.5 | 2.5 | — | — |
| Oleyl Alcohol | — | — | 10 | 10 |
| Diisopropyl Adipate | — | — | 10 | 10 |
| CRODAFOS ™ CES | 2.0 | 2.0 | 5.0 | 5.0 |
| cetearyl alcohol | LT 1.6 | LT 1.6 | LT 4.0 | LT 4.0 |
| dicetyl phosphate | NMT 0.4 | NMT 0.4 | NMT 1.0 | NMT 1.0 |
| ceteareth-10 phosphate | NMT 0.4 | NMT 0.4 | NMT 1.0 | NMT 1.0 |
| Hexylene Glycol | 2 | 2 | — | — |
| Methylparaben | 0.2 | 0.2 | 0.2 | 0.2 |
| Propylparaben | 0.05 | 0.05 | 0.05 | 0.05 |
| Purified Water | q.s. ad | q.s. ad | q.s. ad | q.s. ad |
| | 100% | 100% | 100% | 100% |
| Propellant | About 1:8 blend of propellant to product concentrate | | | |
| Propane/ | NIP- | NIP- | NIP- | NIP- |
| Isobutane/Butane | 70 | 70 | 70 | 70 |
| Description | Acceptable | Acceptable | Acceptable | Acceptable |
| | expanding | stout foam | expanding | stiff foam |
| | foam | | foam | |

TABLE 14

| | Concentration in Product Concentrate (% w/w) | | | |
|---|---|---|---|---|
| | For- | For- | For- | For- |
| | mulation | mulation | mulation | mulation |
| Ingredient | 9 | 10 | 11 | 12 |
| Econazole nitrate | 1% | 1% | 1% | 1% |
| DEGEE (Transcutol P) | 25 | 15 | 20 | 10 |
| Petrolatum | 5.0 | 5.0 | — | — |
| Isopropyl Palmitate | 2.5 | 2.5 | — | — |
| Oleyl Alcohol | — | — | 10 | 10 |
| Diisopropyl Adipate | — | — | 10 | 10 |
| CRODAFOS ™ CES | 10.0 | 10.0 | 10.0 | 10.0 |
| cetearyl alcohol | LT 8 | LT 8 | LT 8 | LT 8 |
| dicetyl phosphate | NMT 2.0 | NMT 2.0 | NMT 2.0 | NMT 2.0 |
| ceteareth-10 phosphate | NMT 2.0 | NMT 2.0 | NMT 2.0 | NMT 2.0 |
| Hexylene Glycol | 2 | 2 | — | — |
| Methylparaben | 0.2 | 0.2 | 0.2 | 0.2 |
| Propylparaben | 0.05 | 0.05 | 0.05 | 0.05 |
| Purified Water | q.s. ad 100% | q.s. ad | q.s. ad 100% | q.s. ad |
| | | 100% | | 100% |
| Propellant | About 1:8 blend of propellant to product concentrate | | | |
| Propane/ | NIP- | NIP- | NIP- | NIP- |
| Isobutane/Butane | 70 | 70 | 70 | 70 |
| Description | Unacceptable | Acceptable | Unacceptable | Acceptable |
| | sputter foam | stout foam | sputter foam | stout foam |

TABLE 15

| | Concentration in Product Concentrate (% w/w) | | | |
|---|---|---|---|---|
| | For- | For- | For- | For- |
| | mulation | mulation | mulation | mulation |
| Ingredient | 13 | 14 | 15 | 16 |
| Ivermectin | 1% | 1% | 1% | 1% |
| DEGEE (Transcutol P) | 25 | 15 | 20 | 10 |
| Petrolatum | 5.0 | 5.0 | — | — |
| Isopropyl Palmitate | 2.5 | 2.5 | — | — |
| Oleyl Alcohol | — | — | 10 | 10 |
| Diisopropyl Adipate | — | — | 10 | 10 |
| CRODAFOS ™ CES | 2.0 | 2.0 | 5.0 | 5.0 |
| cetearyl alcohol | NMT 1.6 | NMT 1.6 | NMT 4.0 | NMT 4.0 |
| dicetyl phosphate | NMT 0.4 | NMT 0.4 | NMT 1.0 | NMT 1.0 |
| ceteareth-10 phosphate | NMT 0.4 | NMT 0.4 | NMT 1.0 | NMT 1.0 |
| Hexylene Glycol | 2 | 2 | — | — |
| Methylparaben | 0.2 | 0.2 | 0.2 | 0.2 |
| Propylparaben | 0.05 | 0.05 | 0.05 | 0.05 |
| Purified Water | q.s. ad | q.s. ad | q.s. ad | q.s. ad |
| | 100% | 100% | 100% | 100% |
| Propellant | About 1:8 blend of propellant to product concentrate | | | |
| Propane/ | NIP- | NIP- | NIP- | NIP- |
| Isobutane/Butane | 70 | 70 | 70 | 70 |
| Description | Acceptable | Acceptable | Acceptable | Acceptable |
| | expanding | expanding | expanding | quick |
| | foam | foam | foam | breaking |
| | | | | foam |

TABLE 16

| | Concentration in Product Concentrate (% w/w) | | | |
|---|---|---|---|---|
| | For- | For- | For- | For- |
| | mulation | mulation | mulation | mulation |
| Ingredient | 17 | 18 | 19 | 20 |
| clobetasol propionate | 0.05% | 0.05% | 0.05% | 0.05% |
| DEGEE (Transcutol P) | 25 | 15 | 20 | 10 |
| Petrolatum | 5.0 | 5.0 | — | — |
| Isopropyl Palmitate | 2.5 | 2.5 | — | — |
| Oleyl Alcohol | — | — | 10 | 10 |
| Diisopropyl Adipate | — | — | 10 | 10 |
| CRODAFOS ™ CES | 2.0 | 2.0 | 5.0 | 5.0 |
| cetearyl alcohol | NMT 1.6 | NMT 1.6 | NMT 4.0 | NMT 4.0 |
| dicetyl phosphate | NMT 0.4 | NMT 0.4 | NMT 1.0 | NMT 1.0 |
| ceteareth-10 phosphate | NMT 0.4 | NMT 0.4 | NMT 1.0 | NMT 1.0 |
| Hexylene Glycol | 2 | 2 | — | — |
| Methylparaben | 0.2 | 0.2 | 0.2 | 0.2 |
| Propylparaben | 0.05 | 0.05 | 0.05 | 0.05 |
| Purified Water | q.s. ad | q.s. ad | q.s. ad | q.s. ad |
| | 100% | 100% | 100% | 100% |
| Propellant | About 1:8 blend of propellant to product concentrate | | | |
| Propane/ | NIP- | NIP- | NIP- | NIP- |
| Isobutane/Butane | 70 | 70 | 70 | 70 |
| Description | Acceptable | Acceptable | Acceptable | Acceptable |
| | quick | quick | quick | quick |
| | breaking | breaking | breaking | breaking |
| | foam | foam | foam | foam |

TABLE 17

| | Concentration in Product Concentrate (% w/w) | | | |
|---|---|---|---|---|
| | For- | For- | For- | For- |
| | mulation | mulation | mulation | mulation |
| Ingredient | 21 | 22 | 23 | 24 |
| oxymetazoline free base | 1% | 1% | 1% | 1% |
| DEGEE (Transcutol P) | 25 | 15 | 20 | 10 |
| Petrolatum | 5.0 | 5.0 | — | — |
| Isopropyl Palmitate | 2.5 | 2.5 | — | — |
| Oleyl Alcohol | — | — | 10 | 10 |
| Diisopropyl Adipate | — | — | 10 | 10 |
| CRODAFOS ™ CES | 10.0 | 10.0 | 10.0 | 10.0 |
| cetearyl alcohol | LT 8 | LT 8 | LT 8 | LT 8 |
| dicetyl phosphate | NMT 2.0 | NMT 2.0 | NMT 2.0 | NMT 2.0 |
| ceteareth-10 phosphate | NMT 2.0 | NMT 2.0 | NMT 2.0 | NMT 2.0 |
| Hexylene Glycol | 2 | 2 | — | — |
| Methylparaben | 0.2 | 0.2 | 0.2 | 0.2 |
| Propylparaben | 0.05 | 0.05 | 0.05 | 0.05 |
| Purified Water | q.s. ad | q.s. ad 100% | q.s. ad | q.s. ad |
| | 100% | | 100% | 100% |
| Propellant | About 1:8 blend of propellant to product concentrate | | | |
| Propane/ | NIP- | NIP- | NIP- | NIP- |
| Isobutane/Butane | 70 | 70 | 70 | 70 |
| Description | Acceptable | Unacceptable | Acceptable | Acceptable |
| | stout foam | sputter foam | stiff foam | stiff foam |

### Example 7 Can Liner Compatibility Testing

Since introduction of a hexane extraction step significantly decreased variability in assay results, a sampling of commercially available can liners were filled with 0.3% foam concentrate and gassed with AP-70 propellant. Three different can sizes were compared to the glass compatibility bottle. The current roflumilast foam 60 gram can was compared to the larger Trivium Cans (PPG-2845 and PPG-8900) that were 53 mm×235 mm cans filled with 275.2 grams concentrate (equivalent to 64 g concentrate for the 60 gram can) and 34.4 grams of AP-70 propellant (equivalent to 8 g or propellant for the 60 gram can). The smaller roflumilast foam 10 gram sample can was filled with 12.0 g concentrate and 2.3 g AP-70 propellant. The bulk concentrate was packaged, and propellant added. The cans were stored inverted and upright at ambient conditions. Bottles were gassed and sent the same days, but were stored upright and horizontal. The assay results for roflumilast, methylparaben and propylparaben are shown in Table 18.

TABLE 18
Results from can liner compatibility study after
ambient storage for over one month.

| | | Average | | | | | |
|---|---|---|---|---|---|---|---|
| | | % | | Average | | | |
| | | Label | | % | | Average | |
| Sample ID | Storage | Claim | | Label | | % Label | |
| and | Orien- | Roflum- | % | Claim | % | Claim | % |
| Description | tation | ilast | RSD | MP | RSD | PP | RSD |
| S200145 | N/A | 100.8 | 0.03 | 100.5 | 0.3 | 98.7 | 1.9 |
| Bulk | | | | | | | |
| Product | | | | | | | |
| S200146 | Upright | 97.2 (B) | 2.0 | 101.9 (B) | 1.8 | 100.5 (B) | 1.8 |
| Trivium can | | 101.2 (M) | | 104.9 (M) | | 103.4 (M) | |
| PPG-8900 | | 98.7 (E) | | 101.5 (E) | | 100.1 (E) | |
| with MPE | Inverted | 93.0 (B) | 3.5 | 96.6 (B) | 3.4 | 96.3 (B) | 4.2 |
| liner Lot | | 89.9 (M) | | 92.3 (M) | | 90.9 (M) | |
| R01-A | | 96.4 (E) | | 98.8 (E) | | 98.7 (E) | |
| S200147 | Upright | 92.5 (B) | 1.9 | 93.7 (B) | 2.3 | 92.4 (B) | 1.7 |
| Trivium can | | 95.8 (M) | | 97.9 (M) | | 95.7 (M) | |
| PPG-2845 | | 93.0 (E) | | 94.7 (E) | | 94.0 (E) | |
| with BPA | Inverted | 95.2 (B) | 1.3 | 96.2 (B) | 1.4 | 94.3 (B) | 1.5 |
| liner Lot | | 92.7 (M) | | 93.9 (M) | | 91.7 (M) | |
| R01-B | | 93.0 (E) | | 93.9 (E) | | 92.4 (E) | |
| S200148 | Upright | 92.6 (B) | 3.0 | 95.2 (B) | 1.3 | 94.0 (B) | 1.7 |
| RM#146427 | | 94.8 (M) | | 96.3 (M) | | 95.9 (M) | |
| Current | | 98.2 (E) | | 97.7 (E) | | 97.3 (E) | |
| ARQ-154 | Inverted | 93.9 (B) | 3.1 | 94.9 (B) | 1.5 | 94.3 (B) | 1.0 |
| 60 g can | | 85.1 (M) | | 95.5 (M) | | 95.2 (M) | |
| with PAM | | 99.5 (E) | | 97.6 (E) | | 96.2 (E) | |
| liner Lot | | | | | | | |
| R01-C | | | | | | | |
| S200149 | Upright | 94.0 (B) | 2.0 | 95.8 (B) | 0.5 | 94.0 (B) | 1.2 |
| RM#146297 | | 92.5 (M) | | 95.6 (M) | | 94.2 (M) | |
| 60 g can | | 96.2 (E) | | 96.6 (E) | | 96.0 (E) | |
| with an | Inverted | 93.1 (B) | 3.7 | 94.7 (B) | 1.2 | 93.8 (B) | 0.8 |
| epoxy | | 93.5 (M) | | 95.4 (M) | | 94.1 (M) | |
| phenolic | | 99.5 (E) | | 97.0 (E) | | 95.2 (E) | |
| liner Lot | | | | | | | |
| R01-D | | | | | | | |
| S200150 | Upright | 98.1 (B) | 2.0 | 95.7 (B) | 1.9 | 90.6 (B) | 1.7 |
| RM#146419 | | 100.1 (M) | | 95.3 (M) | | 91.6 (M) | |
| 10 g can | | 96.1 (E) | | 92.4 (E) | | 88.5 (E) | |
| with an | Inverted | 97.7 (B) | 1.1 | 92.8 (B) | 0.4 | 88.1 (B) | 1.8 |
| epoxy | | 98.6 (M) | | 92.8 (M) | | 87.7 (M) | |
| phenolic | | 96.5 (E) | | 93.5 (E) | | 90.6 (E) | |
| liner | | | | | | | |
| (current) | | | | | | | |
| Lot R01-E | | | | | | | |
| S200151 | Upright | 94.2 (B) | 2.5 | 96.0 (B) | 1.7 | 93.9 (B) | 1.5 |
| Glass | | 90.3 (M) | | 93.8 (M) | | 91.5 (M) | |
| Bottle | | 94.3 (E) | | 96.9 (E) | | 94.0 (E) | |
| Lot R01-F | Horizontal | 92.7 (B) | 1.6 | 94.9 (B) | 0.3 | 92.7 (B) | 0.3 |
| | | 93.5 (M) | | 95.4 (M) | | 92.7 (M) | |
| | | 90.7 (E) | | 94.8 (E) | | 93.1 (E) | |

Variability in the results and the lower than expected values for the glass bottle samples makes it difficult to precisely determine loss to the can liner. However, trends in the data indicate that in terms of retaining near target roflumilast values the epoxy phenolic liner is best, MPE and BPA are similar, but slightly inferior to the epoxy phenolic liner and the current PAM liner is the least compatible liner for the roflumilast foam product. From the data in Table 18 it appears the epoxy phenolic liner may not be compatible with the parabens, especially propyl paraben. If this incompatibility between the preservatives and the epoxy phenolic can liner is confirmed, an overage of roflumilast may be required to compensate for the slight roflumilast loss due to use of the PAM can liner in the primary container for the roflumilast foam.

### Example 8 Roflumilast Foam Final Formulation Experiment

To select the final roflumilast formulation for the manufacture of the three primary stability batches, a matrix of four packaging/propellant combinations is being placed on stability. The four configurations are: 1) current PAM lined can gassed with the AP-70 propellant (The phase 2 IP), 2) current PAM lined can gassed with the AP-48 propellant, 3) epoxy phenolic lined can gassed with AP-70 propellant and 4) epoxy phenolic can gassed with AP-48 propellant. The product concentrate will have the composition shown in Table 2 with the IPP added to the active phase during processing. Target fill weights will be 64.0 grams for the product concentrate and 8.0 grams for the propellant. Forty (40) cans of each of the four configurations will be filled, gassed and placed on stability. Three (3) cans from each configuration will be pulled at each time and tested for assay, impurities, and preservatives.

### Example 9 Storage Stability

The following formulations were prepared and mixed with propellant AP-48 or AP-70 to determine whether a stable foam is formed after storage under ambient conditions for more than 30 days.

TABLE 19

| | Concentration in Product Concentrate (% w/w) | | | |
|---|---|---|---|---|
| | For- | For- | For- | For- |
| | mulation | mulation | mulation | mulation |
| Ingredient | 25 | 26 | 27 | 28 |
| Roflumilast | 0.3% | 0.3% | 0.3% | — |
| DEGEE (Transcutol P) | 25 | 25 | 25 | 25 |
| Petrolatum | 5.0 | 5.0 | 10.0 | 5.0 |
| Isopropyl Palmitate | 2.5 | 2.5 | 5.0 | 2.5 |
| CRODAFOS ™ CES | 2.0 | 2.0 | 2.0 | 2.0 |
| cetearyl alcohol | NMT 1.6 | NMT 1.6 | NMT 1.6 | NMT 1.6 |
| dicetyl phosphate | NMT 0.4 | NMT 0.4 | NMT 0.4 | NMT 0.4 |
| ceteareth-10 phosphate | NMT 0.4 | NMT 0.4 | NMT 0.4 | NMT 0.4 |
| Hexylene Glycol | 2 | 2 | 2 | 2 |
| Methylparaben | 0.2 | 0.2 | 0.2 | 0.2 |
| Propylparaben | 0.05 | 0.05 | 0.05 | 0.05 |
| Purified Water | q.s. ad | q.s. ad | q.s. ad | q.s. ad |
| | 100% | 100% | 100% | 100% |
| Propellant | About 1:8 to 1:6 blend of propellant | | | |
| | to product concentrate | | | |
| Propane/ | AP-48 | AP-70 | AP-70 | AP-70 |
| Isobutane/Butane | | | | |

### Example 10 Evaluation of Foam Quality

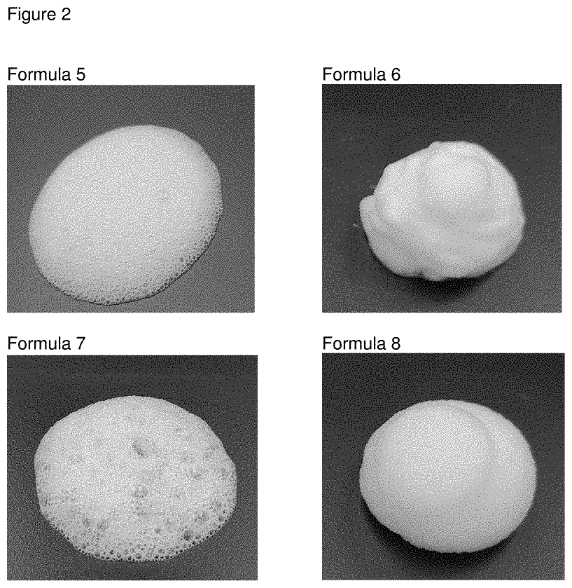

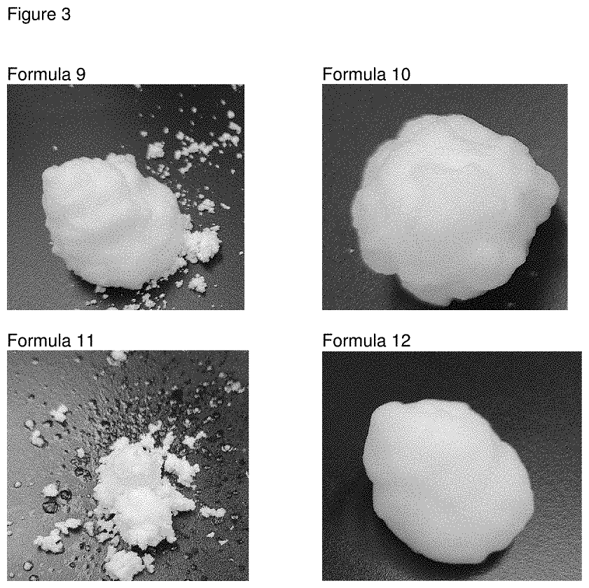

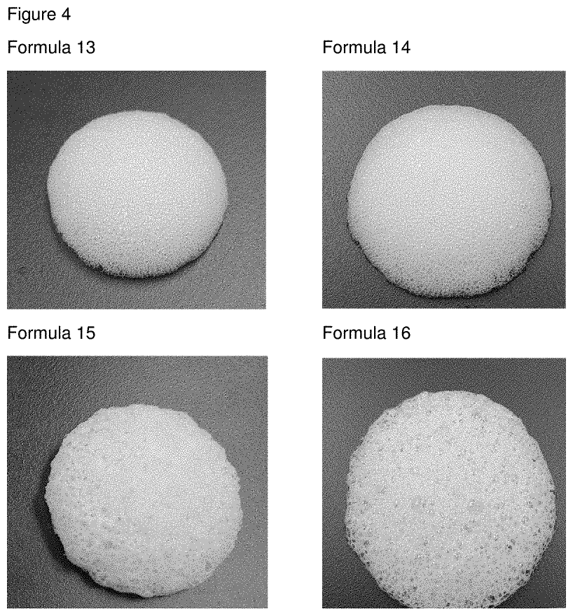

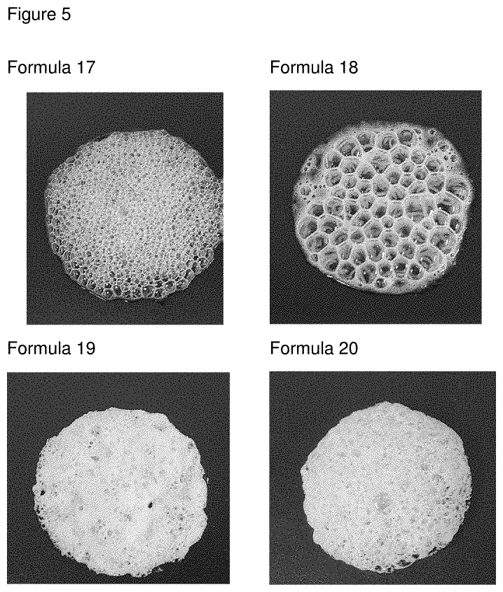

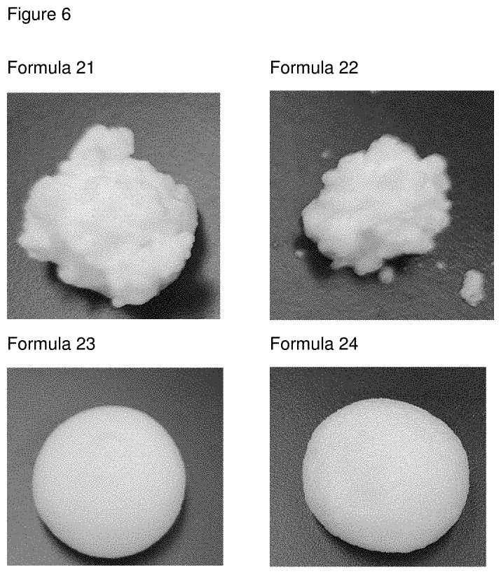

Foams were prepared and assessed using foam quality and foam expansion techniques. Five foam formulations containing an active pharmaceutical ingredient which has low solubility in water were prepared to determine whether a suitable foam product is produced. The APIs included ivermectin, clobetasol proprionate, oxymetazoline free base, ketoconazole and econazole nitrate. The product concentrates containing the APIs were mixed with NIP-70 propellant at a ratio of 88.9% product concentrate to 11.1% propellant. All five of the formulations resulted in a foam product as shown in FIGS. 2-6. The foam quality was assessed visually and by using foam density and foam expansion techniques.

Aerosol can components were prepared according to the following table.

TABLE 20

| Aerosol Components | |
|---|---|
| Utilized | Description |
| 35 mm X 125 mm Can | AER PAM 8460N |
| Stem | S90, 018, (630EQL) Splined |
| Stem Gasket | S90, BUNA, B1785 Non-Food Grade |
| Spring | S90, STEM, 020, Stainless Steel |
| Body | S90, Housing Inverted W/Tail Piece 4 Slot |
| Mounting Cup | Aluminum Spherical Cup, Epon T/B |
| Dip Tube | STD, LLDPE, 0.122 Nat 5K FT |

The sample variable tolerances were as follows.

TABLE 21

| Variable | Minimum | Target | Maximum |
|---|---|---|---|
| Foam concentrate Fill Weight | 60.8 g | 64.0 g | 67.2 g |
| Propellant Fill Weight | 7.6 g | 8.0 g | 8.4 g |
| Crimp Height | 0.208” | 0.210” | 0.212” |
| Crimp Diameter | 1.065” | 1.07” | 1.075” |

### Example 11

Four formulations were tested for each of five different low water soluble actives including ivermectin, clobetasol propionate, oxymetazoline, ketoconazole, and econazole nitrate.

Samples were prepared for each variable. Each can was filled with 64 g of the product concentrate containing the API, followed by crimping. The cans were subsequently pressurized with 8 g of NIP-70 propellant. The propellant was filled manually using a burette system followed by weight analysis of individual samples. A +/−5% range from the target weights was deemed acceptable. No sample deviated more than 3% from the target values. The finished aerosol products utilized 75% of the specified can's brim filled capacity. The finished cans were tested for leaks by submerging in a water bath at 55° C. for 10 minutes. No leaks were detected during visual inspection of the submerged cans. The finished cans were shaken by hand for no more than 10 seconds and allowed to rest for at least 2 days to ensure complete mixing of the propellant and product concentrate.

The samples were studied using visual analysis to determine the presence or absence of a foam after dispensing. A foam was defined as the visual presence of multiple bubbles sharing a minimum of 1 liquid film wall which may be broken when agitated by an external force. The foams were visually analyzed immediately after dispensing and again five minutes after dispensing. Formulations 5-8, 10, 12-21, 23, and 24 were found to produce acceptable foams immediately after dispensing and 5 minutes after dispensing. The acceptable foams were smooth white or off-white foams having uniform bubbles and were able to support their own weight. The foam half-life was more than 60 seconds. The resulting foams are shown in FIGS. 2-6. All five APIs produced foams for all of the formulations that were tested. A wide range of foam structures were observed for the different formulations showcasing the range of foams which can be produced using the tested APIs. The foam structures can be optimized for specific indications.

| | | Representation |
|---|---|---|
| Formulation Number | Appearance | in FIGS. |
| Formulation 5 | Acceptable expanding foam | 2 |
| Ketoconazole | | |
| Formulation 6 | Acceptable stout foam | 2 |
| Ketoconazole | | |
| Formulation 7 | Acceptable expanding foam | 2 |
| Ketoconazole | | |
| Formulation 8 | Acceptable stiff foam | 2 |
| Ketoconazole | | |
| Formulation 9 | Unacceptable sputter foam | 3 |
| Econazole nitrate | | |
| Formulation 10 | Acceptable stout foam | 3 |
| Econazole nitrate | | |
| Formulation 11 | Unacceptable sputter foam | 3 |
| Econazole nitrate | | |
| Formulation 12 | Acceptable stout foam | 3 |
| Econazole nitrate | | |
| Formulation 13 | Acceptable expanding foam | 4 |
| Ivermectin | | |
| Formulation 14 | Acceptable expanding foam | 4 |
| Ivermectin | | |
| Formulation 15 | Acceptable expanding foam | 4 |
| Ivermectin | | |
| Formulation 16 | Acceptable quick breaking foam | 4 |
| Ivermectin | | |
| Formulation 17 | Acceptable quick breaking foam | 5 |
| Clobetasol proprionate | | |
| Formulation 18 | Acceptable quick breaking foam | 5 |
| Clobetasol proprionate | | |
| Formulation 19 | Acceptable quick breaking foam | 5 |
| Clobetasol proprionate | | |
| Formulation 20 | Acceptable quick breaking foam | 5 |
| Clobetasol proprionate | | |
| Formulation 21 | Acceptable stout foam | 6 |
| oxymetazoline free base | | |
| Formulation 22 | Unacceptable sputter foam | 6 |
| oxymetazoline free base | | |
| Formulation 23 | Acceptable stiff foam | 6 |
| oxymetazoline free base | | |
| Formulation 24 | Acceptable stiff foam | 6 |
| oxymetazoline free base | | |

1. An aerosol foam comprising an active pharmaceutical ingredient with water solubility below 60 mg/l, cetearyl alcohol, dicetyl phosphate, and ceteareth-10 phosphate in an oil in water emulsion; and a propane/isobutane/butane propellant blend, wherein said active pharmaceutical ingredient with water solubility below 60 mg/l is not roflumilast.

2. The aerosol foam according to claim 1, wherein said oil in water emulsion has a viscosity of 4,000-11,000 cP.

3. The aerosol foam according to claim 1, wherein said propellant and oil in water emulsion are in a ratio of about 1:8 to 1:6.

4. The aerosol foam according to claim 1, wherein said aerosol foam is emitted from a container but collapses after application to a subject's skin.

5. The aerosol foam according to claim 1, wherein said active pharmaceutical ingredient is selected from the group consisting of ketoconazole, econazole nitrate, ivermectin, clobetasol propionate, calcipotriene, halobetasol propionate, tazarotene, oxymetazoline free base and desonide.

6. The aerosol foam according to claim 1, further comprising at least one component selected from the group consisting of hexylene glycol and diethylene glycol monoethyl ether.

7. The aerosol foam according to claim 6, wherein said diethylene glycol monoethyl ether is in an amount of 25% w/w to 35% w/w.

8. The aerosol foam according to claim 6, further comprising at least one additional component selected from the group consisting of a solvent, moisturizer, surfactant or emulsifier, polymer or thickener, preservative, antioxidant, sequestering agent, stabilizer, buffer, pH adjusting solution, skin penetration enhancer, film former, dye, pigment, and fragrance.

9. The aerosol foam according to claim 6, further comprising an additional active agent selected from the group consisting of anthralin, azathioprine, tacrolimus, coal tar, methotrexate, methoxsalen, salicylic acid, ammonium lactate, urea, hydroxyurea, 5-fluorouracil, propylthiouracil, 6-thioguanine, sulfasalazine, mycophenolate mofetil, fumaric acid esters, corticosteroids, corticotropin, vitamin D analogues, acitretin, tazarotene, cyclosporine, resorcinol, colchicine, adalimumab, ustekinumab, infliximab, bronchodialators, and antibiotics.

10. The aerosol foam according to claim 1, wherein said cetearyl alcohol, dicetyl phosphate, and ceteareth-10 phosphate are in an emulsifier blend in an amount of 2 to 4% by weight of the total composition.

11. The aerosol foam according to claim 10, wherein said emulsifier blend is in an amount of 2% by weight of the total composition.

12. The aerosol foam according to claim 1, further comprising water in an amount of 55 to 70% by weight of the total composition.

13. The method according to claim 1, wherein said active pharmaceutical ingredient is in an amount of 0.05 to 2% by weight of the total composition.

14. The aerosol foam according to claim 1, wherein said aerosol foam comprises said active pharmaceutical ingredient, white petrolatum, isopropyl palmitate, cetearyl alcohol, dicetyl phosphate, ceteareth-10 phosphate, hexylene glycol, diethylene glycol monoethyl ether, methylparaben, propylparaben, purified water and a propane/isobutane/butane propellant blend.

15. A method for treating a patient with an inflammatory skin condition, comprising topically administering an aerosol foam comprising an active pharmaceutical ingredient with water solubility below 60 mg/l, cetearyl alcohol, dicetyl phosphate, and ceteareth-10 phosphate in an oil in water emulsion; and a propane/isobutane/butane propellant blend to a patient in need thereof, wherein said active pharmaceutical ingredient with water solubility below 60 mg/l is not roflumilast.

16. The method according to claim 14, wherein said aerosol foam further comprises hexylene glycol and/or diethylene glycol monoethyl ether.

17. The method according to claim 16, wherein said diethylene glycol monoethyl ether is in an amount of 25% w/w to 35% w/w.

18. The method according to claim 14, wherein said composition is administered 1-2 times per day.

19. The method according to claim 15, wherein said patient is suffering from a proliferative, inflammatory and/or allergic dermatoses.

20. The method according to claim 19, wherein said proliferative, inflammatory and allergic dermatoses is selected from the group consisting of psoriasis (vulgaris), eczema, acne, Lichen simplex, Lichen sclerosus, Prurigo nodularis, sunburn, pruritus, alopecia areata, hypertrophic scars, discoid lupus erythematosus, and pyodermias.

21. The method according to claim 19, wherein said patient is suffering from an inflammatory dermatoses.

22. The method according to claim 21, wherein said patient is suffering from atopic dermatitis.

23. The method according to claim 15, wherein said aerosol foam does not include hexylene glycol.

24. A method for solubilizing an active pharmaceutical ingredient with a water solubility below 60 mg/l in an aerosol foam, comprising
combining an active pharmaceutical ingredient with a water solubility below 60 mg/l, cetearyl alcohol, dicetyl phosphate, and ceteareth-10 phosphate in an oil in water emulsion, and
combining the oil in water emulsion with a propane/isobutane/butane propellant blend to produce an aerosol foam,
wherein said active pharmaceutical ingredient with water solubility below 60 mg/l is not roflumilast.

25. An aerosol foam comprising an active pharmaceutical ingredient with water solubility below 60 mg/l, cetearyl alcohol, dicetyl phosphate, ceteareth-10 phosphate and diethylene glycol monoethyl ether in an oil in water emulsion, and a propane/isobutane/butane propellant blend, wherein said oil in water emulsion has a viscosity of 4,000-11,000 cP, wherein said foam does not include hexylene glycol, wherein said diethylene glycol monoethyl ether is in an amount of 25% w/w to 35% w/w, wherein said aerosol foam is emitted from a container but collapses after application to a subject's skin, and wherein said aerosol foam has a foam half-life of more than 60 seconds.
